(19) United States
(12) Patent Application Publication
(10) Pub. No.: US 2025/0000064 A1
(43) Pub. Date: Jan. 2, 2025

(54) MEDICINAL LEECH STORAGE AND DISINFECTION DEVICE

(71) Applicant: ISTANBUL MEDIPOL UNIVERSITESI TEKNOLOJI TRANSFER OFISI ANONIM SIRKETI, Beykoz, Istanbul (TR)

(72) Inventors: Halil Ibrahim INANÖZ, Beykoz, Istanbul (TR); Ali Timuçin ATAYOGLU, Beykoz, Istanbul (TR)

(21) Appl. No.: 18/712,035

(22) PCT Filed: Nov. 1, 2022
(86) PCT No.: PCT/TR2022/051219

§ 371 Date: May 21, 2024

(30) Foreign Application Priority Data
Nov. 24, 2021 (TR) 2021/018402

(51) Int. Cl. A01K 61/40; A01K 63/00; A01K 63/04; A61L 2/10

(52) U.S. Cl. CPC A01K 61/40; A01K 63/003; A01K 63/04; A61L 2/10

(57) ABSTRACT

Disclosed is a medicinal leech storage and disinfection device for enabling easy and standardized storage, health monitoring, and disinfection processes of medicinal leeches and reducing the water usage in leech cultivation and personnel load in follow-up procedures.

### TECHNICAL FIELD

The invention relates to a medicinal leech storage and disinfection device for enabling easy and standardized storage, health monitoring, and disinfection processes of medicinal leeches and reducing the water usage in leech cultivation and personnel load in follow-up procedures.

### BACKGROUND OF THE INVENTION

Leeches have been used since ancient times, especially for phlebotomy in the treatment of diseases for medical purposes. Hirudotherapy is found in the earliest inscriptions of Egypt, Persia, Europe, China, and Anatolia, and it is frequently included in the treatment protocols of Ibni Sina, which is called Avicenna by Galen and Europeans. Medicinal leeches used in hirudotherapy (Leech Therapy) applications are usually supplied from medicinal leech farms by cargo during certain seasons of the year and are usually stored in glass jars in departments, rooms, or cabinets separated in practitioner clinics. Antibiotic support and/or disinfection of leeches are performed in the application protocols before the application. Due to the metabolism of leeches, when certain pH, oxygen, and temperature conditions are met and when the storage water is renewed with chlorine-free (purified or spring) water approximately every 2 weeks, it can be stored for 10-15 weeks depending on the size of the leech. In Turkey, clinics work without stock with regular orders according to the number of leeches used or with stock if storage area and appropriate personnel can be provided. Storing medicinal leeches in clinics is costly due to the pH of the water to be stored, the difficulty of temperature and oxygen monitoring, the need for personnel, and the high use of water.

It is known that medicinal leeches are directly supplied and used without stock due to production in completely closed facilities in the USA and European countries and prescription sales of medicinal leeches in pharmacies. In Turkey, medicinal leeches provided with orders such as 10-100-1000 leeches are stored in a 10-liter glass jar. Approximately 50 medicinal leeches can survive for 2 weeks with 7 liters of water. When it is predicted that 6-8 leeches are used in one session, it is possible to apply 1000 leeches per month. For 1000 leeches, 420 liters of purified water is required for the filling of 20 10-liter glass jars 3 times a month, first filling, cleaning in the 2nd week, and cleaning in the 4th week. This process, as well as the assignment of 1 personnel for monitoring and processing of water for pH, oxygen, and temperature, also constitutes an additional cost. It is aimed to solve these problems with the invention.

Currently, it is known that antibiotic prophylaxis is generally applied to patients in medicinal leech applications in hospitals. Throughout history, the problem of disinfection by changing water with ceramic, metal, or glass jars has been tried to be temporarily eliminated. Today, in the medicinal leech application training of the Ministry, medicinal leech disinfection methods are described in two ways; the first method is performed with 12.5 ppm hydrochloric acid for 10 minutes+washing+distilled water (storage). The second method is; 0.3 ppm methylene blue for 20 minutes+washing+25 ppm antibiotic prophylaxis for 2 hours+washing+1 day with distilled water.

The methods used are performed by the practitioner or assistant health personnel, and additional information is required for solution preparation and disinfection. Therefore, these usage methods are not practical and economical. After the medicinal leeches provided by the invention are placed in the device, an automatic and effective disinfection process is performed according to the selected method.

Leeches are invertebrate parasites that can move in 3 different ways, live in both water and land, and breathe with their skin. Medicinal leeches, on the other hand, are leeches that give various enzymes to the animal to suck blood and that are separated from the animal so that they normally suck blood without causing infection. While the movements of the leeches in the water are carried out by fluctuation, they move by using their front and back tugs for their movements on land or the surface. They perform this move through their ability to vacuum. In the clinical environment, while transferring leeches from one environment to another (examination room, disinfection process, etc.), there are inner chambers included in the invention as an apparatus that has a surface that they cannot hold by vacuum to facilitate their collective transfer, and that they cannot pass through their holes even though they are invertebrate. The inner chambers have two purposes, the first is that they cannot be vacuumed because they are perforated (if the leeches make a vacuum, they can escape by climbing), and they cannot pass through because they are small perforated. The second purpose of the inner chambers is to provide easy transportation to the disinfection process or inspection area. While it will waste time to take 50 leeches from an aquarium with 250-300 leeches under normal conditions, with the invention, it is easier to take 50 leeches from it and carry them with the chambers live in the inner chambers.

The object of the invention is to monitor the health status of medicinal leeches used in clinics, to ensure that disinfection processes and storage are carried out easily and in a standard manner. Another object of the invention is to reduce the personnel load in water use and follow-up procedures during the storage of medicinal leeches. In addition, the invention mechanically solves the disinfection method and application processes, which have not been proven to be effective in the clinic and which vary according to the expert, within the technological and scientific framework.

The invention is a special aquarium that can be easily used in clinics where medicinal leeches are applied and are used for the storage of leeches for leech treatment and the disinfection of the leeches used. It enables the preparation of the water in the system according to the desired mode and the monitoring of the characteristics of the system with the internal purification system, water motor, sensors, heating-cooling device, light, and smart system equipment.

Each chamber has a camera, motion sensor, and heat sensor on the side and upper cover. The system detects the movements and vitality of leeches with artificial intelligence.

The invention provides savings and convenience by seeing the pH, temperature, and oxygen monitoring of the water on the internal screen and smart devices with the application, cleaning and reusing the water used, and adding the necessary amount of new water to the system. It reduces the water and personnel load, making the problem of medicinal leech storage in the clinic the easiest for users. In addition, it is to carry out the disinfection process of medicinal leeches in an easy, economical, and effective way according to the selected method. During the disinfection process, solution preparation reduces the personnel load during the application and follow-up stages. The disinfection method selected by placing the leeches in the inner chamber is applied by the device.

There is a vacuum system in the aquarium that can be raised and lowered in the area where the inner chamber is installed. When this system is switched on, it raises the apparatus on the ground of the chamber and opens the vacuum outlets. This vacuum apparatus is connected to the ground system by the magnet and prevents the movement of the inner chamber.

While the leeches are alive and healthy, they act as fluctuations in the water, swimming, sticking outside the water, and advancing according to the environment they are in. The dead leeches cannot move because they have lost these characteristics. Since the water balance is disrupted, they are swollen and immobile. It is possible to understand this visually. Generally, dead leeches fall from the surface they are adhered to and come to the bottom of the container they are in, and may be displaced depending on the current. In the storage area of the invention, there is a vacuum system for the exit of dead leeches at the middle level of each chamber. This system is activated by the joining of the vacuum system parts on the tank and the aquarium ground with the magnet and the software detecting the dead leech. The vacuum system, which has a metal surface with a magnet, is connected to the outer surface. The system rises and lowers with the help of the motor. The vacuum system draws dead leeches and debris from the vacuum inlets while the vacuum system is open.

Digital cameras, heat, and motion sensors are located on the upper side and side of the storage area to monitor the growth stages of the leeches. It is analyzed with artificial intelligence with the data obtained from digital cameras and sensors. The growth, vitality, and movement status of the leeches are determined accordingly.

Leeches sometimes live in watery environments and sometimes in environments without water. There is a rest area attached to the ground with a magnet attached to the middle line of the tank so that the leeches desired to be grown in the aquarium can be kept in an anhydrous environment. Thanks to the grip (rest) bar in the invention, it is used for the leeches to live attached to this bar. By adjusting the amount of water in the chamber, it is ensured that the leech can hold on to the upper part of the bar and stand in an anhydrous environment.

The structural and characteristic features and all the advantages of the invention will be understood more clearly with the figures and the detailed description with reference to these figures given below therefore, the evaluation should be made by taking these figures and the detailed description into consideration.

### FIGURES FOR DESCRIBING THE INVENTION

FIG. 1: A perspective view of medicinal leech storage and disinfection device

FIG. 2: A top view of medicinal leech storage and disinfection device

FIG. 3: A perspective device of the inner chamber of the medicinal leech storage and disinfection device

FIG. 3a: A view of the front side of the inner chamber

FIG. 3b: A perspective view of the upper side of the inner chamber

FIG. 3c: A perspective view of the chamber transport apparatus of the inner chamber

FIG. 3d: A perspective view of the vacuum system chamber apparatus

FIG. 3e: A perspective view of the open state of the vacuum system chamber apparatus

FIG. 3f: A perspective view of the closed state of the vacuum system chamber apparatus

FIG. 4: A front view of medicinal leech storage and disinfection device

FIG. 4a: A front view of the sensor camera and lighting system

FIG. 4b: A top view of the upper cover

FIG. 5: A perspective view of the vacuum system

FIG. 6: A front view of the grip (rest) bar

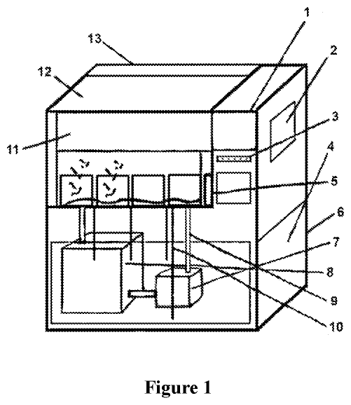

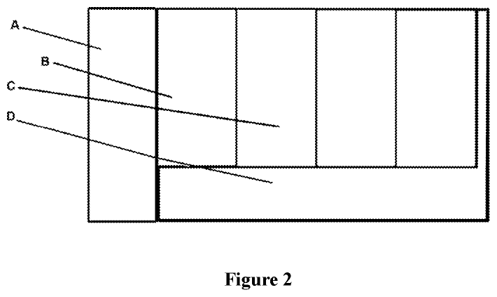

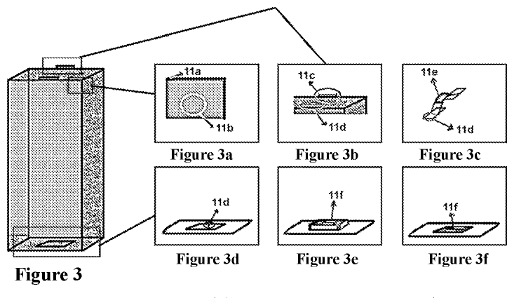

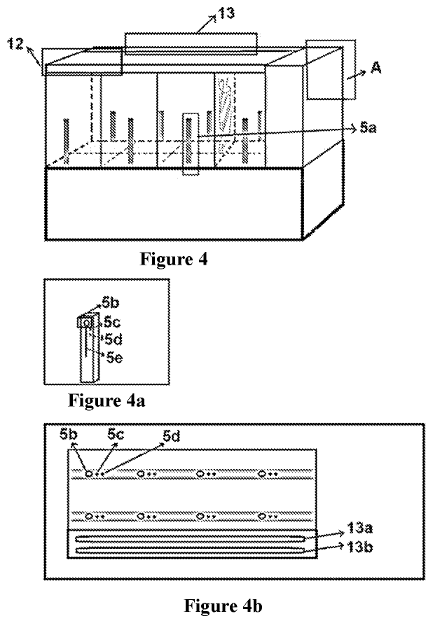

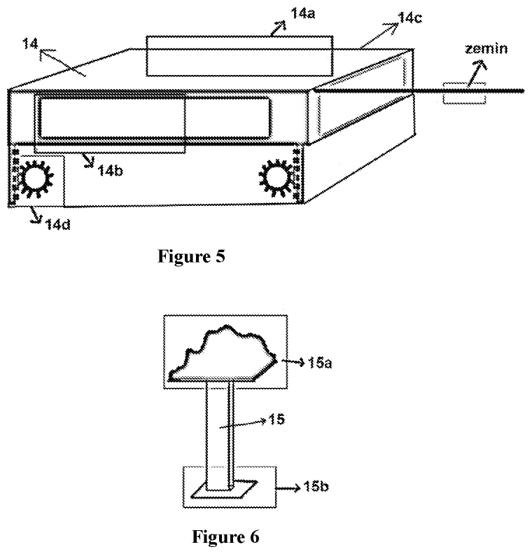

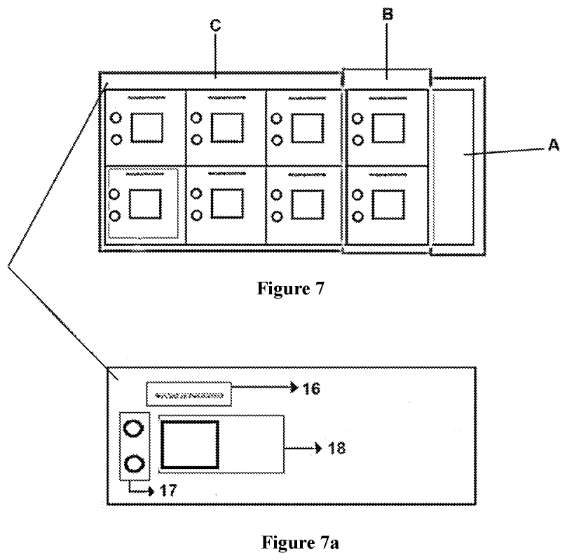

FIG. 2: A top view of medicinal leech storage and disinfection device

FIG. 7a: A top view of the inner chambers

### REFERENCES FOR DESCRIBING THE INVENTION

1. Screen
2. Hardware cover
3. Keys
4. Motor area side cover
5. Sensor area
   5a. Sensor, camera, and lighting system
   5b. Camera
   5c. Motion sensor
   5d. Heat sensor
   5e. Light system
6. Motor cover front cover
7. Water and air motor
8. Purification device
9. Water installation
10. Air installation
11. Inner chambers
   11a. Frame
   11b. Perforated outer surface
   11c. Transport apparatus hook
   11d. Magnet
   11e. Chamber transport apparatus
   11f. Vacuum system
12. Upper cover
13. Light system area
   13a. UV light system
   13b. Lighting system
14. Vacuum system
   14a. Vacuum system upper surface
   14b. Vacuum system inlet section
   14c. Vacuum system outlet section
   14d. Motor
15. Grip (rest) bar
   15a. Rest area
   15b. Grip bar metal ground
16. Air system outlet section
17. Water inlet outlet section
18. Vacuum system ground apparatus
   A. Hardware and solution loading area
   B. Disinfection area
   C. Storage area
   D. Lighting and UV system area

### DESCRIPTION OF INVENTION

The invention is described as a medicinal leech storage and disinfection device without any limiting effect in the detailed description.

The said invention comprises:
A hardware and solution area (A),
A disinfection area (B) where the purification device and water and air motor are located,
A storage area (C) where the storage area of medicinal leeches is located,
A lighting and UV system area (D).

The invention comprises:
A screen (1) showing the values of temperature, pH, and oxygen ratio of the water in the device and monitoring data,
A hardware cover (2) that stores the equipment in which it is located,
Keys (3) on the front of the device, which enable manual adjustment of the values of water such as temperature and pH.
A motor area side cover (4) to the side of the water and air motor,
A sensor area (5) where the sensors that enable the detection of the temperature, pH, and oxygen ratio of the water are located,
A sensor, camera, and lighting system (5a) recording the data on the side of each inner chamber (11) and the upper cover (12),
A camera (5b) located on the side of each inner chamber (11) and the upper cover (12), which detects the movement of the leeches in the area it is in,
A motion sensor (5c) located on the side and upper cover (12) of each inner chamber (11), which detects the vitality of the leeches in the area it is located in,
A heat sensor (5d) located on the side of each inner chamber (11) and on the upper cover (12), which allows the temperature of the water to be measured,
A lighting system (5e) enabling the device to be illuminated in its environment,
A motor area front cover (6) of the front side of the water and air motor,
Water and air motor (7) that renews or cleans, cools or discharges water according to sensor information,
The purification device (8) for cleaning the contaminated water,
A water installation (9) that provides the transmission of cleaned or cooled water to leeches,
An air installation (10) that provides the transmission of fresh air to leeches,
Inner chambers (11) that are located on the upper part of the device and are the storage area of medicinal leeches,
A metal or plastic frame (11a) forming the outside of the inner chambers (11),
A perforated outer surface (11b) on the outside of the inner chambers (11), which allows the leeches to adhere,
A transport apparatus hook (11c) located on the upper side of the inner chambers (11) and used for mounting the chamber transport apparatus (11e),
A magnet (11d) that prevents it from moving by fixing the inner chambers (11) to the area,
A chamber transport apparatus (11e) that allows easy transportation of inner chambers (11) by the user,
A vacuum system (11f), which allows the inner chambers (11) to rise and fall from the area,
An upper cover (12), which covers the inner chambers (11) and allows air to breathe thanks to its small holes,
a light system area (13) in which the lighting system (13b) and the UV light system (13a) are located and the disinfection process is carried out, which has RGB lights and also UV lights, and in which UV disinfection can be performed at the time (at night, etc.) set through the application or internal software,
A UV light system (13a) that disinfects with UV lights in the area of the light system (13),
A lighting system (13b), which is the lighting task within the area of the light system (13),
A vacuum system (14) for the output of dead leeches at the middle level of each chamber in the storage area (C),
A vacuum system outer surface (14a), which is magnetized in the metal structure on the upper surface of the vacuum system (14),
A vacuum system inlet section (14b), which is the opening part of the vacuum system (14),
A vacuum system outlet section (14c), which is the outlet for removing dead leeches from the aquarium,
A motor (14d) used for vacuuming water and drawing leeches,
A grip (rest) bar (15), which allows the leeches to hold on in an anhydrous environment,
A rest area (15a) that allows leeches to rest by holding on and to stand in a thirsty environment,
A metal ground (15b) that provides adhesion with a magnet,
An air system outlet section (16), where the air is discharged from the storage area (C),
A water inlet outlet section (17), where the water inlet and outlet to the system are made,
A vacuum system, which enables the vacuum system to adhere to the ground, consists of parts of the vacuum system ground apparatus (18).

The invention consists of hardware and solution area (A), disinfection area (B), storage area (C), and lighting and UV system area (D) sections. In the invention, there is a water and air motor (7) with a purification system in the lower half of the device, and it is a medicinal leech storage and disinfection device with a control area in the upper half and a partitioned aquarium area with a hardware area and solution addition part in the upper half There are 8 chambers in the aquarium area and 2 chambers at the bottom of these chambers are used as disinfection units. Necessary solutions are added from the solution addition area.

When connected to the water installation, the device purifies the water inside and fills the aquarium storage area at a rate of ⅔. It can detect the temperature, pH, and oxygen ratio of the water in the aquarium with the sensors in the sensor area (5). The sensors used to measure the physical and chemical properties of the water are located before the water pumped into the aquarium enters the aquarium and before the water drawn from the aquarium enters the purification process. The purpose here is to record the current status of the system and the values of the water pumped to balance it and to present it to the user. According to the sensor information seen through the application, the decision to renew or clean, cool, or drain the water is made by the user. The tracking of the amount of leech and the size of the leech placed in each chamber is recorded according to the user's information on adding and removing leech. It reports the number of leeches used according to the months and recommends the amount of stock required for the next period. The data can be viewed on the screen on the device or reported on the smart device.

The camera (5b), motion sensor (5c), and heat sensor (5d) are located on the upper and side parts of the storage area (C) and the side and upper cover (12) of each inner chamber (11) to follow the growth stages of the leeches. The sensor, the camera, and the lighting system (5a) include the camera (5b), the motion sensor (5c), and the heat sensors (5d). It is analyzed with artificial intelligence with the data obtained from the cameras (5b) and sensors. The growth, vitality, and movement status of the leeches are determined accordingly. In order to determine the operating time of the vacuum system (14), there is a motion sensor (5c) and a heat sensor (5d) in the storage area (C). With these sensors, the movements of the leeches in the environment are detected. When there is a stationary object in the environment, the sensors transmit information to the system. Thus, the vitality of the leeches is determined. The system detects the movements and vitality of leeches with artificial intelligence.

In the hardware and solution area (A), the chemicals required to be added for the disinfection process are added to the device as a concentrate and according to the set program, the device dilutes and uses the necessary amount of solution and it is the area that includes the necessary parts for the computer such as the chip, battery, screen and is internally available in the system.

The aquarium area consists of two independent sections. The first of these is the storage area (C) that follows the characteristics of the water in the water, replaces the water with clean water when necessary, and keeps it in balance, and the second is the disinfection area (B) that can pump and discharge the water containing solution. Leeches are placed in these areas with an average of 50 leech capacity chambers and can be removed.

The inner chambers (11) have a standard, small perforated outer surface (11b) and have a metal or plastic frame (11a). Its purpose is to collectively remove or transfer the leeches to other units. There are inner chambers (11) for the collective and rapid transfer of leeches. The water of the disinfection feature of these tanks belongs to the tank and only the water from the storage tanks is common. In the section specified as hardware and solution loading area (A), there are entries where we can add 3 separate solutions. Solutions to be used in disinfection are added here. There is a vacuum system (11f) in the aquarium that can be raised and lowered in the area where the inner chamber (11) is installed. When this vacuum system (11f) is opened, it raises the apparatus on the ground of the chamber and opens the vacuum outlets. This vacuum apparatus is connected to the ground system by the magnet (11d) and prevents the movement of the inner chamber.

The light system area (13) includes the lighting system (13b) and the UV light system (13a). In this area, the UV disinfection process is carried out at the time (night, etc.) set through the application or internal software with RGB lighting light and also UV light.

Thanks to the invention, the leech disinfection process and leech storage function are performed. The water to be used for the disinfection of the leeches is prepared in the 2-chamber section located at the bottom of the device. The device fills and empties the disinfection chamber by diluting the required solution with water in accordance with the steps according to the method selected on the screen (1). The disinfection process in accordance with the selected method is delivered to the inner chambers (11) in the disinfection area (B), which can pump and discharge the water from the purification device (8), the relevant amount of solution and the water motor and the water containing the solution in the disinfection area. Since the inner chambers (11) have a perforated outer surface (11b), a vacuum cannot be made and the leeches are transferred more easily during the transfer. In addition, thanks to the inner chambers (11), the leeches are located in different sections, they are easily carried by the chamber transport apparatus (11e) during use and it is easy to take the desired number of leeches. The chamber transport apparatus (11e) is fixed and used by the transport apparatus hook (11c).

The leech storage function is transmitted to the inner chambers (11) thanks to the clean water air installation (10) in the water and air motor (7), and thanks to the water installation (9), it is transmitted to the storage area (C) that keeps the water in balance by replacing it with clean water when necessary. Thanks to the application, it reports the number of leeches disinfected by months and recommends the required stock amount for the next period. It shows all follow-up data on the screen (1) or is reported as a pdf over the smart device via the application.

In the storage area (C) of the invention, there is a vacuum system (14) for the exit of dead leeches at the middle level of each chamber. There is a magnetic vacuum system outer surface (14a) with a metal structure on the upper surface of this system. This system is activated by the joining of the vacuum system parts on the tank and the aquarium ground with the magnet and the software detecting the dead leech. When the leech outlet is provided, the vacuum system opening when activated consists of the inlet part (14b), the discharge pipe, the motor (14d), and the vacuum system outlet part (14c), which is the outlet part of the leeches. The vacuum system inlet section (14b) is located in the storage area (C) at the middle level of each chamber and provides an outlet for vacuumed water and dead leeches to move away from the environment. The discharge pipe is used to allow dead leeches from the vacuum system inlet section (14b) to travel to the vacuum system outlet section (14c). The motor (14d) is used to vacuum the water and draw the leeches. The vacuum system outlet section (14c) is the outlet used to remove dead leeches from the aquarium. In addition, there are geared motors (14d) that enable the system to be opened and closed. The vacuum system, which has a metal surface with a magnet, is connected to the outer surface (14a). The system rises and lowers with the help of the motor (14d). The vacuum system (14) draws dead leeches and debris from the vacuum inlets while the vacuum system is open.

Leeches sometimes live in watery environments and sometimes in environments without water. There is a rest bar (15) attached to the metal ground (15b) with a magnet attached to the centerline of the chamber so that the leeches desired to be grown in the aquarium can be kept in an anhydrous environment. Thanks to the grip (rest) bar (15) in the invention, it is used for the leeches to live attached to this bar. The grip (rest) bar (15) can be removed and attached. By adjusting the amount of water in the chamber, it is ensured that the leech can rest and stand in the waterless environment by holding on to the rest area (15a) on the top of the bar.

Each compartment of the inner chambers (11) divided into compartments in the storage area (C) has an air system outlet section (16) and a water inlet outlet section (17). On the lower side of these compartments, there is a vacuum system ground apparatus (18) that allows it to adhere to the ground.

The technical features mentioned in each claim are followed by a reference number, these reference numbers are used only to facilitate understanding of the claims so they should not be considered to limit the scope of any of the elements indicated by these reference numbers for illustrative purposes.

It is obvious that a person skilled in the art can make use of similar structures and/or apply this structure to other similar fields used in the relevant art. Therefore, it is obvious that such structures will lack the criterion of exceeding the innovation and especially the state of the art.

1. Medicinal leech storage and disinfection device comprising:
a screen showing values of temperature, pH, and oxygen ratio of water in the device and monitoring data,
a hardware cover that stores equipment in which it is located,
keys on a front side of the device, which enable manual adjustment of the values of water such as temperature and pH,
a motor area side cover of the side of a water and air motor,
a motor area front cover of the front side of the water and air motor,
wherein the water and air motor (7) that renews or cleans, cools, or discharges water according to sensor information,
a purification device for cleaning the contaminated water,
a water installation for transmitting the cleaned or cooled water to leeches,
an air installation for transmitting fresh air to leeches,
inner chambers located on an upper part of the device and having a storage area for medicinal leeches and a solution loading area,
a metal or plastic frame forming the outside of the inner chambers,
a perforated outer surface on the outside of the inner chambers for adhering the leeches,
a transport apparatus hook located on the upper side of the inner chambers and used for mounting the chamber transport apparatus,
a magnet that prevents it from moving by fixing the inner chambers to the area,
a chamber transport apparatus that allows easy transportation of inner chambers by the user,
a vacuum system which allows the inner chambers to rise and fall from the area,
an upper cover which covers the inner chambers and allows it to take the air through its small holes,
a light system area in which a lighting system and a UV light system are located and the disinfection process is carried out, which has RGB lights and also UV lights, and in which UV disinfection can be performed at the time (at night, etc.) set through the application or internal software,
wherein the UV light system disinfects with UV lights in the light system area,
wherein the lighting system having the lighting task in the light system area,
a vacuum system for the output of dead leeches at the middle level of each chamber in the storage area,
a vacuum system outer surface, which is magnetized in the metal structure on the upper surface of the vacuum system,
a vacuum system inlet section which is the opening part of the vacuum system,
a vacuum system outlet section which is the outlet for removing dead leeches from the aquarium,
a motor used for vacuuming water and drawing leeches,
an air system outlet section where the air is discharged from the storage area,
a water inlet outlet section where the water inlet and outlet to the system are made, and
a vacuum system ground apparatus for adhering the vacuum system to the ground.

2. A medicinal leech storage and disinfection device according to claim 1, comprising:
a sensor area for detecting the temperature, pH, and oxygen ratio of the water by the sensors,
a sensor, camera, and lighting system for recording the data on the side of each inner chamber and the upper cover,
a camera for detecting the movement of leeches in the area in which it is located,
a motion sensor for detecting the vitality of the leeches in the area it is located in,
a heat sensor for measuring the temperature of the water, and
a light system for illuminating the device in its environment.

3. A medicinal leech storage and disinfection device according to claim 1, comprising:
a grip (rest) bar for holding the leeches in an anhydrous environment,
a rest area that allows leeches to rest by holding on and to stand in a thirsty environment, and
a metal ground for adhering it with the magnet.

4. A medicinal leech storage and disinfection device according to claim 1, comprising:
the hardware and solution area where the chemicals required to be added for the disinfection process are added to the device as concentrated and the hardware parts are included internally,
disinfection and storage area including the purification device and water and air motor,
a storage area including the storage area of medicinal leeches,
lighting and UV system area in which the system is illuminated and disinfection processes are carried out.

5. A medicinal leech storage and disinfection device according to claim 1, wherein the user decides to renew or clean, cool, or discharge the water in the device according to the user sensor information by working with the application and with the application.

6. A medicinal leech storage and disinfection device according to claim 1, comprising sensors that operate through the application and keep records of the current status of the system and the values of the water pumped to balance it and provide the user with the follow-up of these data.

7. A medicinal leech storage and disinfection device according to claim 1, comprising inner chambers that are easily transported and prevent the leeches from vacuuming with its perforated structure.

8. A medicinal leech storage and disinfection device in accordance with claim 1, wherein there is a camera, motion sensor, and heat sensor on the side of each inner chamber and upper cover.

9. A medicinal leech storage and disinfection device according to claim 1, wherein the vacuum system rises and lowers thanks to the motor and ensures the removal of dead leeches from the system.

10. A medicinal leech storage and disinfection device according to claim 1, wherein it can detect the movements and vitality of leeches with artificial intelligence through sensors.

11. A medicinal leech storage and disinfection device according to claim 1, comprising an air system outlet section, a water inlet outlet section in each compartment of the storage area, and vacuum system ground apparatus at the bottom.
